(19) United States
(12) Patent Application Publication
(10) Pub. No.: US 2013/0101462 A1
(43) Pub. Date: Apr. 25, 2013

(54) METHOD AND SYSTEM FOR DISINFECTING AND STERILIZING HOLLOW BODIES

(71) Applicant: Gernot Keil, Braunweiler (DE)

(72) Inventor: Gernot Keil, Braunweiler (DE)

(73) Assignee: KHS GmbH, Dortmund (DE)

(21) Appl. No.: 13/808,359

(22) PCT Filed: May 24, 2011
(86) PCT No.: PCT/EP2011/002585

§ 371 Date: Jan. 4, 2013

(30) Foreign Application Priority Data
Jul. 9, 2010 (DE) 10 2010 026 759.7

(51) Int. Cl. A61L 2/20

(52) U.S. Cl. CPC A61L 2/208
USPC 422/33; 422/28; 422/292

(57) ABSTRACT

A hollow body sterilizing or disinfection method using a sterilizing agent, such as periacetic acid or hydrogen peroxide, introduced into a body at ambient pressure from a vaporizer includes supplying the sterilizing agent in the vaporizer as dry sterilizing agent vapor, the dry sterilizing agent vapor comprising vapor with a sterilizing agent quantity or vapor density that is at most equal to and preferably less than saturated vapor density such that upon introduction of the sterilizing agent vapor into the hollow body and upon subsequent expansion of the sterilizing agent vapor from a pressure in the vaporizer to ambient pressure, condensate formation occurs substantially only on a surface of the hollow body.

The invention relates to a method for the sterilisation and/or disinfection of hollow bodies using a vaporised sterilising medium or agent according to the generic term of patent claim 1 and a system for the sterilisation and/or disinfection of hollow bodies using a vaporised sterilising medium or agent according to the generic terms of patent claim 14.

Sterilising agent in the meaning of the invention means quite generally a fluid and vaporisable treatment medium or agent, which is suitable for the sterilisation and/or disinfection of hollow bodies, for example peracetic acid or hydrogen peroxide (H₂O₂) in each case in aqueous solution and at a sufficiently high concentration, preferably at a concentration of at least 20 percent by weight (hereinafter called simply “peracetic acid” or “hydrogen peroxide”).

Hollow bodies in the meaning of the invention are, inter alia, quite generally packaging materials, as used for the packaging of products, in particular also fluid or viscous products, or blanks for the manufacture of such packaging materials, such as for example containers, bottles, cans or pots made of metal, glass, plastic or other materials or combinations of materials, in particular PE bottles, yoghurt pots, or preforms for the manufacture of plastic containers or bottles by blow-moulding etc.

Vapour and in particular sterilising agent vapour too in the meaning of the invention means a set of particles which, before condensation, behaves like an ideal gas, i.e. it forms a gas phase, in which the vapour is completely dry, from which the vapour however is nonetheless able to condense. The vapour or sterilising agent vapour can be under-saturated. The vapour density then lies below the saturation vapour mass or density which is determined by the temperature which dictates the condensation point of the vapour. The vapour or sterilising agent vapour can however also just be saturated. Then the volume of the vapour contains just enough vaporised mass or quantity, namely vaporised saturated vapour mass or quantity “m_sätt”, that it does not condense. A small temperature drop of the volume in which the vapour is found, then however leads to the condensation of sterilising agent vapour or mass until the saturated vapour mass or density is reached once again.

Wet vapour consists of dry vapour and a moist phase. Wet vapour is thus super-saturated and carries more mass than it can carry in comparison to the saturated vapour mass. The moist phase is the part of the vapour which has already condensed. Wet vapour can thus also be termed aerosol.

In the meaning of the invention, the expression “in the main” means deviations from the particular precise value by +/−10%, and preferably by +/−5%.

Processes for the sterilisation and/or disinfection of hollow bodies, for example for the sterilisation and/or disinfection of bottles or similar containers, are generally known, whereby the sterilising agent is often introduced finely dispersed into a carrier gas, for example into sterile air, and then the carrier gas containing the sterilising agent after heating and vaporising the sterilising agent, is fed via a vapour pipe into the relevant hollow body. On the internal surface of the hollow body wall, a condensate of the sterilising agent forms, and from said condensate then for example by activation oxygen atoms or radicals and OH molecules are released to kill microorganisms or germs.

Also known are processes and systems for sterilisation and/or disinfection (DE 10 2004 059 346 A1, DE 103 14 687 A1), in which the condensate formation of the sterilising agent used, from the sterilising vapour, occurs suddenly in a previously evacuated sterilisation chamber. The condensate formation is here restricted to adiabatic processes in which, to generate the sudden condensation in a short time, i.e. within 50 to 300 milliseconds, large vapour masses have to flow. This can be achieved in a vacuum and where the vapour at the place of its expansion, i.e. at entry into the sterilisation chamber is restricted as little as possible in its expansion behaviour, thus there are practically no flow or current limits. In the transfer of these known processes to the sterilisation and/or disinfection of hollow bodies at normal or ambient pressure, however, when the vapour expands from the initial pressure, which is for example 3 bar, to the normal or ambient pressure, there is necessarily a mist formation (wet vapour) or premature condensate formation in the wet vapour with the crucial drawback that only with an extremely high consumption of vaporised sterilising agent could the surfaces to be disinfected and/or sterilised be reliably completely covered with the sterilising agent and thus would the quality of sterilisation and/or disinfection sought be achievable. The known processes require a sterilisation chamber which can be evacuated. These processes are therefore not intended and also not suitable for the sterilisation of hollow bodies at ambient pressure.

The purpose of the invention is to describe a process and a system with which an improved sterilisation and/or disinfection of hollow bodies, of high quality (sterilisation/disinfection level) and in a simple manner, in particular also at ambient or atmospheric pressure is possible. To resolve this task, a process is formed according to patent claim 1. A system is the subject of patent claim 14.

One particularity of the invention is that the vaporised sterilising agent (sterilising agent vapour), which is preferably hydrogen peroxide or peracetic acid vapour, is introduced into the particular hollow body as dry or saturated vapour such that no premature condensation or mist formation occurs within the hollow body, and instead the condensation occurs only on the hollow body wall to be disinfected and/or to be sterilised. To this end, the sterilising agent is vaporised in the at least one vaporiser such that it forms a gas phase even after introduction into the hollow body, or the vapour density is below the saturated vapour density even after introduction into the hollow body and after the subsequent expansion to normal or ambient pressure.

A further particularity of the invention consists of the introduction of the sterilising agent vapour into the particular hollow body without a carrier gas, which is possible in particular due to the sterilising agent vapour in the at least one vaporiser being supplied as dry vapour or at most as saturated vapour at a vapour pressure which is clearly above ambient pressure.

To carry out the process according to the invention, the disinfecting or sterilising agent is thus vaporised in the vaporiser in such a way that a gas phase forms and the vapour density is below the saturated vapour mass or density. The vaporised disinfecting or sterilising agent or the sterilising agent vapour is introduced into the particular hollow body preferably without the use of a carrier gas (e.g. air), i.e. free of carrier gas and/or in such a way that condensation only or in the main only occurs on the wall of the hollow body. Conditioned stainless steel or aluminium surfaces have proven to be suitable as surfaces coming into contact with the sterilising agent vapour. The sealing materials and structural materials in the vaporiser valves can be made of Viton, polyethylene, perbunan, teflon, nylon, polyethylene or silicon. The disinfecting agent is measured into the vaporiser by a disinfectant dispenser. The process according to the invention uses at least one sterilising agent vaporiser in which the sterilising agent (sterilising medium), for example hydrogen peroxide or peracetic acid (or another fluid sterilising agent) is vaporised such that a gas phase arises from the sterilising agent in the vaporiser. If the selected temperature of the vaporiser is sufficiently high, a partial pressure of the sterilising agent occurs which is higher than air pressure.

At 20% H₂O₂ (hydrogen peroxide) and at a vaporiser temperature, i.e. the temperature at which the vaporiser is operated and which corresponds approximately to the wall temperature in the vaporiser, of 120° C., a total vapour pressure of 1.65 bar arises. At 30% H₂O₂, with the same vaporiser temperature, a total vapour pressure of 1.48 bar arises and with a vaporiser temperature of 140° C., total vapour pressures of 3.1 bar and 2.79 bar respectively arise, as shown in Table 1 below. All percentages given relate to the concentration “KN” of the H₂O₂ in the hydrogen peroxide and are percentages by weight.

The temperature which corresponds to the lowest temperature “T_min” of the vaporiser surface accessible to the sterilising agent is to be regarded as the vaporising temperature. If the temperature profile of the surface of the vaporiser is very marked, i.e. if there is a big variation between the minimum surface temperature “T_min” and the maximum surface temperature “T_max”, then inside the vaporiser a maximum vapour pressure arises which is defined by “T_min” and the saturated vapour mass “m_sätt” corresponding to “T_min”. Good vaporisers in the meaning of this invention have only a small temperature variation along their surface, one which is better than ±1 K, so that T≈T_min≈T_max.

If the vaporiser is made of aluminium or stainless steel, it can be operated at up to around 150° C. without high hydrogen peroxide decomposition rates occurring in intervals of a few minutes. To achieve this, it is however essential for the vaporiser walls accessible to the peroxide either to be passivated by oxidative processes or for the vaporiser itself to be passivated by the operation with the sterilising agent.

Vaporiser pressures can be generated which pre-tension the sterilising agent sufficiently, for example to 3 bar at 140° C. so that the sterilising agent drives itself out of the vaporiser when an outlet valve is opened to release the sterilising agent vapour. The system according to the invention can thus be operated without a carrier gas.

The decomposition of the sterilising agent can be recognised in that, where the quantity of sterilising agent introduced into the vaporiser at a concentration “KN” (for example 20 percent by weight) at vaporiser temperature T corresponds at maximum to the saturation mass or quantity, the pressure in the vaporiser does not aim for a saturation value but rises steadily above the saturated vapour pressure which is dictated by the vaporiser temperature and the concentration “KN”. As described above, for 20% H₂O₂ at a temperature of 120° C. a partial pressure of 1.65 bar can arise. If the vaporiser is completely evacuated before the injection of the fluid hydrogen peroxide, i.e. close to 0 mbar, and if then a mass “m_sätt” is injected into the vaporiser, which corresponds to the saturated vapour mass, then a vapour pressure of 1.65 bar maximum can arise as the saturated vapour pressure. If this pressure is not fully reached for an injection mass “m_sätt”, then either somewhere on the inside wall of the vaporiser there is an under-cooled area of wall, the temperature of which is below the vaporiser temperature “T”, or alternatively the hydrogen peroxide is not yet fully vaporised. If the vaporiser pressure however rises above the saturated vapour pressure, then this is an indication that the hydrogen peroxide is decomposing. This behaviour also applies for other meta-stable sterilising agents, such as for example peracetic acid, although here the saturated vapour pressures have different values. If the vaporiser is not completely evacuated, but an air pressure “p_Luft” remains, then a pressure increase “p+p” arises if an injection mass “m_sätt” was injected, no decomposition occurs and the mass “m_sätt” is completely vaporised, whereby “p” is the saturated vapour pressure corresponding to injection mass “m_sätt”.

TABLE 1
Saturated vapour pressures “P” of hydrogen peroxide
vapours at concentration “KN”

| 120° C. | | 130° C. | |
|---|---|---|---|
| KN [%] | P [bar] | KN [%] | P [bar] |
| 10 | 1.78 | 10 | 2.51 |
| 20 | 1.65 | 20 | 2.32 |
| 30 | 1.48 | 30 | 2.09 |
| 140° C. | | 150° C. | |
| KN [%] | P [bar] | KN [%] | P [bar] |
| 10 | 3.36 | 10 | 4.48 |
| 20 | 3.1 | 20 | 4.14 |
| 30 | 2.79 | 30 | 3.73 |

In the practical operation of a vaporiser, the stability of the concentration “KN” of the vapour (no or extremely low decomposition rate) of a sterilising agent can thus be set and/or monitored via the stability of the vapour pressure or the pressure of the vapour phase, provided that the sterilising agent is completely vaporised and the quantity of the mass to be vaporised in the vaporiser remains below the saturated vapour mass “m_sätt”. It has been shown, from operating vaporisers for hydrogen peroxide, that with well-conditioned surfaces inside the vaporiser, the vapour pressure does not rise for a period of 10 minutes at an operating temperature of 120° C. and 50% H₂O₂. Even at a temperature of 140° C. and a concentration of 30% H2O2, the vapour pressure is stable for 5 minutes. Expediently, with vaporisers made of stainless steel or aluminium and hydrogen peroxide concentrations of 30%, an operating temperature of 150° C. should not be exceeded. At this temperature, in practice a pressure course was measured which, starting from the saturated vapour pressure, increased by around 10% per minute.

However, considerably higher pressure increases can also occur which can be due on the one hand to the operating temperature of the vaporiser being too high and on the other to the condition of the surfaces which are in contact with the hydrogen peroxide. Experience shows that hot surfaces coming into contact with hydrogen peroxide or peracetic acid must be well-conditioned to keep the decomposition rates of the meta-stable peroxide molecules low.

As explained above, the decomposition rate can be monitored and/or set indirectly via the pressure rise arising once the saturation pressure has been reached. Every mass “m_inj” of an introduced or injected quantity of hydrogen peroxide at concentration “KN” leads via the ideal gas law to a defined vapour pressure, namely:

p*V=m*R_{—}i*T,

where p is the pressure arising at injection of a mass m in a volume V which is maintained at a temperature T. The saturation pressure “p_sätt” can arise as a maximum pressure provided that a mass “m” is injected which corresponds to the saturation mass “m_sätt” and provided that the mass is completely vaporised. “RJ” is the specific gas constant which results in the case of water from the molar gas constant “R_m” (R_m=8,315 J/(mol*K)) divided by the molar mass of the gas (subject to an ideal gas behaviour) at 462 J/kg*K and in the event of pure hydrogen peroxide at 245 J/kg*K, whereby T is given in K (Kelvin). For aqueous hydrogen peroxide mixtures, the weighted mean of both values must be taken.

A good conditioning or good passivation for areas of aluminium or stainless steel surfaces coming into contact with the hydrogen peroxide or peracetic acid is formed by an oxide layer. This can be provided either by chemical oxidation or by chemical pre-oxidation and in the subsequent treatment by the operation of the vaporiser with hydrogen peroxide or peracetic acid. Inadequately conditioned surfaces of a hot vaporiser are further conditioned during their operation with peroxides so that, after a conditioning time of 10 to 100 hours, surface conditions arise which behave passively against hot peroxides and do not generate decomposition. In this conditioning time, peroxide vapours which have saturation levels of 10 to 90% are cyclically applied to all surfaces which are hot and come into contact with peroxides. Low saturation levels are then selected here if high decomposition constants arise, for example at a pressure rise of 10% every 10 seconds. High saturation levels are then selected if low decomposition constants arise, for example at a pressure rise of 10% every 2 minutes.

Essential characteristics of the process according to the invention or the system according to the invention lie inter alia in that:
the introduction of the sterilising agent into the particular hollow body occurs without a carrier gas, and/or
the condensation of the sterilising agent occurs only or in the main only on the hollow body wall, thus when the sterilising agent is introduced into the hollow body and when the sterilising agent vapour expands to normal or ambient pressure, no condensation occurs in the sterilising agent vapour, and/or
the introduction of the sterilising agent vapour in the particular hollow body occurs with isothermal or in the main isothermal expansion characteristics, and/or
the treatment, i.e. the disinfection and/or sterilisation of the hollow body occurs at normal or ambient pressure, and/or prior warming of the hollow body is not absolutely necessary, and/or
the sterilisation agent deposited as condensate on the hollow body or its wall is activated automatically, for example at least with the aid of the heating arising during condensation.

Further developments, benefits and application possibilities of the invention arise also from the following description of examples of embodiments and from the figures. In this regard, all characteristics described and/or illustrated individually or in any combination are categorically the subject of the invention, regardless of their inclusion in the claims or reference to them. The content of the claims is also an integral part of the description.

The invention is explained in further detail below with reference to the figures. The following are shown:

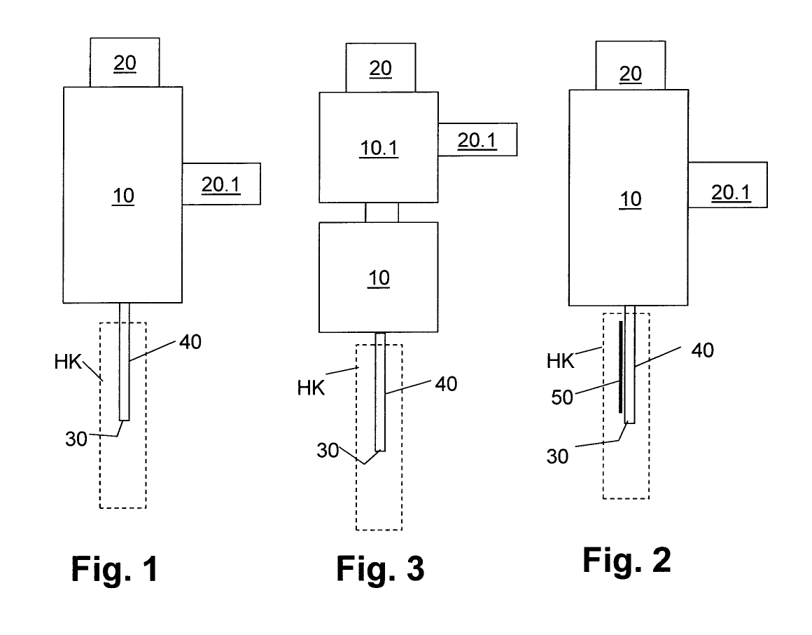

FIG. 1 in a very simplified schematic representation, a treatment station with a vaporiser for the sterilisation and/or disinfection of hollow bodies with the sterilising agent vapour;

FIG. 2 an illustration as FIG. 1, but with a further embodiment of the invention;

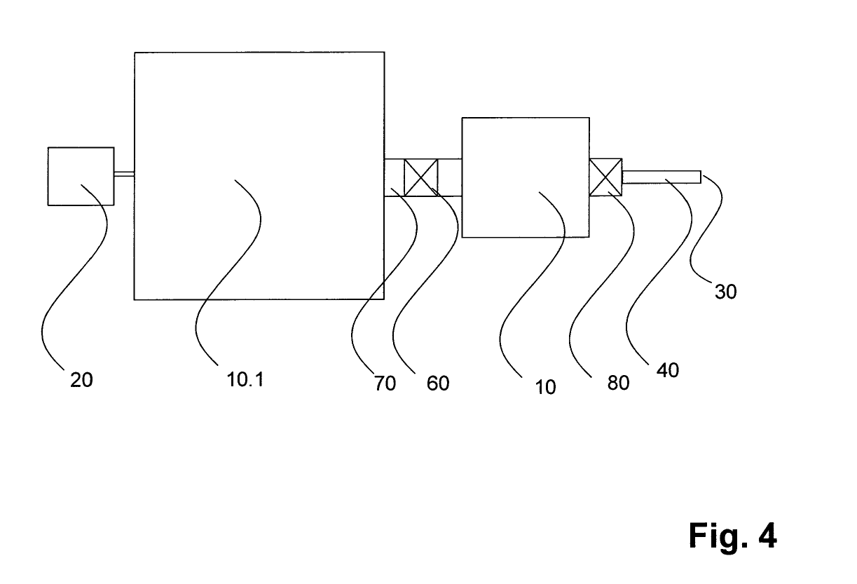

FIG. 3 a treatment station with a vaporiser and with an additional vaporiser or pre-vaporiser;

FIG. 4 the treatment station of FIG. 3 in a schematic block diagram or functional diagram;

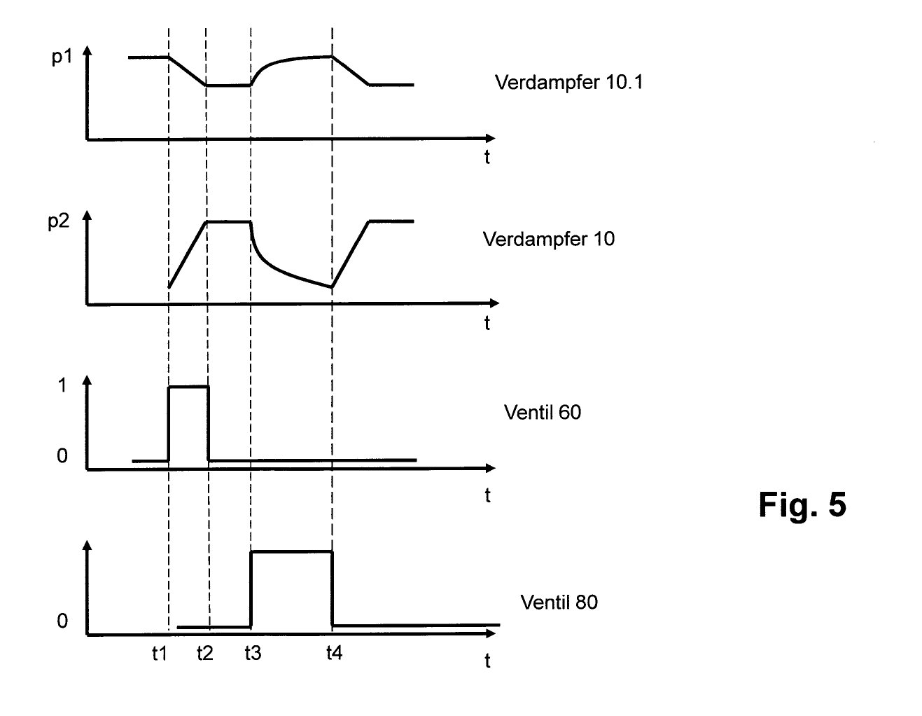

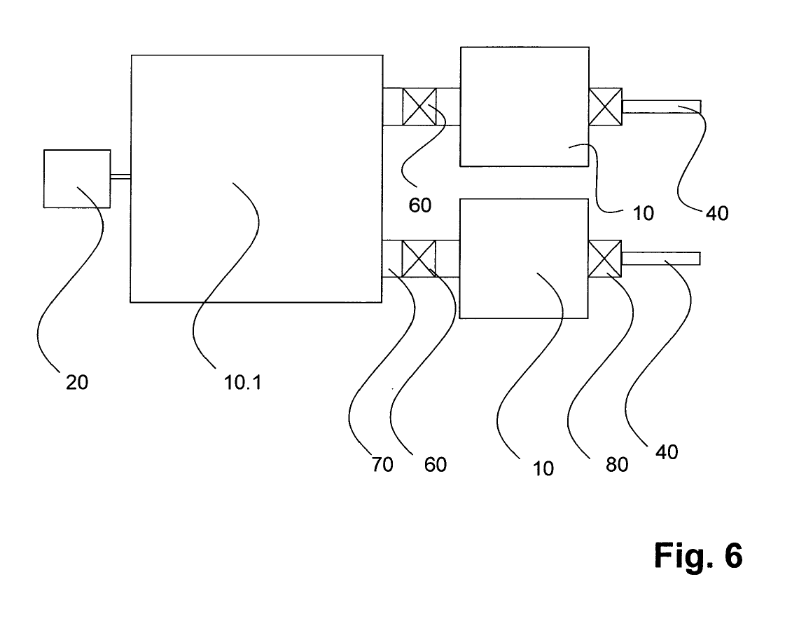

FIG. 5 graphs which show the chronological course of the vapour pressure in the vaporisers of the treatment station of FIGS. 3 and 4 and the condition of the valves of this treatment station;

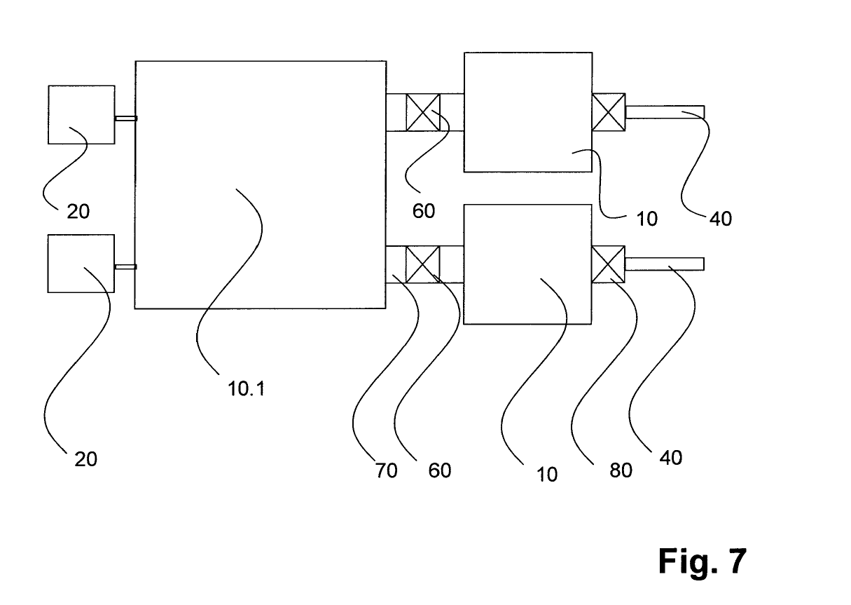

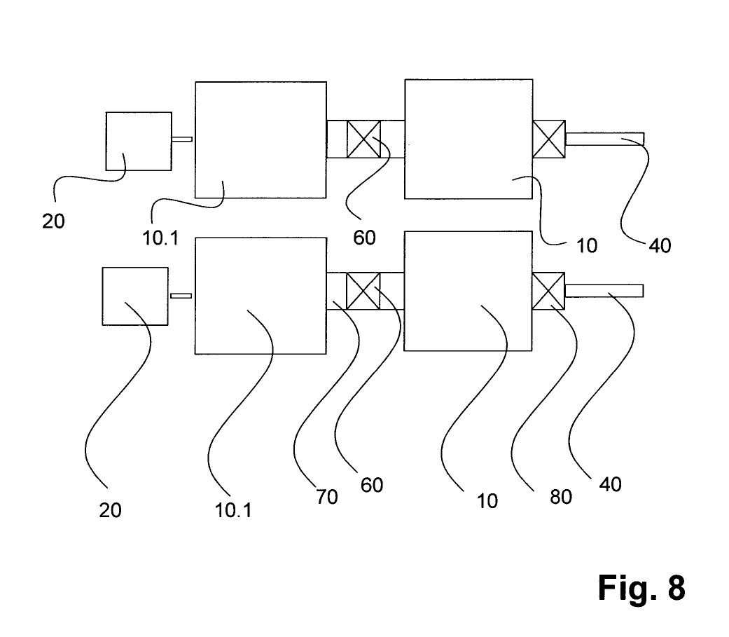

FIGS. 6-8 in illustrations similar to FIG. 4, further embodiments of treatment stations of the system according to the invention.

One example of the system according to the invention for the disinfection and/or sterilisation of hollow bodies “HK” is shown in FIG. 1. The vaporiser called 10 there is supplied from a metering unit 20 with just enough sterilising agent for the sterilising agent to vaporise completely. To vaporise the sterilising agent, it is pushed by the metering unit 20 on a dosing point into the vaporiser 10. The metering unit 20 can be a metering pump 20.1 which introduces measured quantities of sterilising agent at the dosing point into the vaporiser 10. The metering unit 20 can however also consist of a fluid container which receives the sterilising agent pre-tensioned with a gas or a membrane, whereby by the time-controlled opening of a valve on the fluid container or on the dosing point, in each case a certain quantity of sterilising agent is introduced into the vaporiser. In the vaporiser 10, the injected sterilising agent then vaporises. It is apparent that the sterilising agent vaporises faster, the smaller the measured quantity or the bigger the vaporiser surface covered by the fluid medium and the higher the operating temperature of the vaporiser 10. On practical embodiments of vaporisers with just one dosing point and a volume of 10 l, a vaporisation rate of up to 10 g per minute is measured at a temperature of 120° C. This rate can be increased further if on the one hand a number of dosing points are used or on the other a number of vaporisers are used.

In a vaporiser volume of 10 I, a mass of hydrogen peroxide of 17 g (at 30% H₂O₂) can be vaporised until saturation of the vapour at a temperature of 413 K. If the mass is completely vaporised, a pressure of 2.8 bar has arisen, subject however to the vaporiser having first been evacuated. If the vaporiser was not evacuated, the partial pressure of the air would need to be added to the partial or vapour pressure of the hydrogen peroxide vapour, whereby according to the invention, the operation of the vaporiser 10 with air is not necessary, but instead the introduction of the vaporised sterilising agent (sterilising agent vapour) free of air or another carrier gas, into the particular hollow body “HK” is possible. Likewise, the evacuation of the vaporiser by the injection of the sterilising agent is not absolutely essential because by the cyclical injection of sterilising agent and by the cyclical removal of sterilising agent vapour and the mixing of the sterilising agent and the sterilising agent vapour with an air phase possibly existing in the vaporiser, the air phase is automatically increasingly removed from the vaporiser 10.

To nonetheless free the vaporiser of air more rapidly, an evacuation device can be disposed on the vaporisers 10 and 10.1 which can consist of a sealing water pump, a water-ring air pump, and a booster and piston pump or a similar vacuum device.

The vapour phase which builds up a partial pressure of 2.8 bar in a vaporiser volume of 10 litres, corresponds to a volume of 28 litres of vapour at normal pressure, i.e. a vapour volume of 28 bar litres. The vapour which is being pre-tensioned in vaporiser 10 can flow out of the vaporiser by opening an outlet valve which connects the vaporiser 10 to a vapour pipe 40 which can be inserted in the particular hollow body and which forms an outlet on the lower end, provided that the pressure of the vaporiser 10 is above the external air pressure. Of the 28 bar litres of vapour volume, therefore, the usable vapour volume is therefore 19 bar litres, as following the release of this volume, a pressure corresponding to the ambient pressure is reached. At the latest after the release of the vapour volume with which the pressure in the vaporiser has fallen to ambient pressure, the repeated injection of sterilising agent must be started in the vaporiser 10.

With a vapour volume of 18 bar litres, 360 preforms (50 cm̂3 content) or 18 PET bottles with a volume of 1 l can be completely filled, whereby the complete filling of the volume of the particular hollow body “HK” is not absolutely necessary for surface disinfection because the vapour, upon injection into the hollow body “HK”, mixes with the air in the hollow body and is then diluted.

The hot sterilising agent vapour flows via the outlet valve or discharge opening 30 of the vaporiser into the hollow body “HK” to be disinfected, whereby the hollow body wall temperature determines the condensation or dew point. In a similar way as in the vaporisers 10 or 10.1, for which, from data in the literature and from calculations about the ideal gas law, which for pressures up to approx. 10 bar and temperatures up to 160° C. for water vapour and hydrogen peroxide vapour can be taken as a good approximation, a saturated vapour density of 1.7 kg/m̂3 for 20% hydrogen peroxide at 120° C. can be calculated, the saturated vapour density in the hollow body “HK” to be disinfected at a temperature of 20° C. is approx. 17 g/m̂3 or 17 mg/litre. The saturated vapour density in the hollow body “HK” to be sterilised is thus around 2 orders of magnitude lower than the vapour density of the vapour flowing out of the vaporiser 10 or the discharge opening 30. Even if a vapour volume of 15 bar litres were taken from the vaporiser 10, the vapour density of the sterilising agent vapour emerging from the vaporiser is still around a factor of 40 higher than the saturated vapour density in the hollow body “HK”, whereby the entire usable vapour volume can be used for the condensation on the hollow body wall of the particular hollow body “HK”.

The condensation of the vapour occurs everywhere where the vapour density exceeds the saturated vapour mass or density. Starting from the discharge opening 30 of the vaporiser 10 and the direction of flow of the vapour into the hollow body “HK” to be sterilised, no condensate will form in the first phase of the outflow as the hollow body “HK” is vapour-free at this point.

It is even possible for the vapour to reach the hollow body wall condensation-free. Then it continues to behave in the manner of a gas. If however, with a hollow body temperature of 20° C., the vapour density rises over 17 mg/litre, the hydrogen peroxide starts to condense.

A substantial particularity of the invention lies in the fact that the sterilising agent vapour reaches the hollow body wall without—or in the main without—prior condensation. Indeed, if condensation were to commence on the route from the discharge opening 30 to the hollow body wall, then either mist would form, such mist being very fine drops of condensate, or however large drops of the sterilising agent. In both cases, however, a condensate precipitation on the hollow body wall would only be extensive if a disproportionate amount of condensate can precipitate, thus if the condensation process were for a prolonged period and/or a corresponding large quantity of sterilising agent vapour were introduced. Moreover, the drops do not precipitate on the condensation cores, but instead somewhere on the hollow body wall.

There is thus a clear difference between the condensation of an aerosol (which consists of already partially formed condensate drops) and the condensate formation from a sterilising agent vapour which behaves like a gas and contains no moist phase, as the invention describes. The sterilising agent vapour is always deposited on condensation cores, which are also primarily the micro-organisms to be killed. If vapour is allowed to condense, the micro-organisms are always hit. If wet vapour or aerosols are allowed to condense, the micro-organisms are only hit if a sufficient mass of the sterilising agent hits the hollow body wall, so that a closed sterilising agent film can form there.

Thus if vapour of a defined density passes over a surface, the surface temperature of which defines a lower saturated vapour density than the density of the vapour flowing past, a forced condensation of the vapour on this surface is achieved. As the vapour flows out of the vaporiser without a carrier medium, a forced condensation without a carrier medium or carrier gas is achieved which is regarded as a substantial characteristic of the method of this invention.

Because of the condensation, condensation heat is released in the condensate which heats up the condensate or sterilising agent film. As, in the method according to the invention, vapour with a high vapour density moves over the surfaces to be sterilised—high here means preferably a vapour density increased by a factor of two compared with the temperature of the hollow body “HK” to be disinfected, said density being in comparison with the saturated vapour density of the hollow body wall—large quantities of condensate are formed and thus a rapid heating of the condensate or sterilising agent film is achieved, the temperature of which is determined by the condensate mass, by the condensation speed (precipitation of the mass “dm” per interval of time “dt”) and by the heat conduction properties of the hollow body “HK” to be disinfected.

In the initial phase of condensation, which lasts only around 50 to 200 mm, the droplets formed on the hollow body wall are very small and the condensate film is not closed. Certainly, the condensate droplets form on the condensation cores and they are the germs or micro-organisms to be killed. For these condensate drops, a large part of the heat formed can be rapidly diverted into the hollow body wall so that the drops only heat up moderately. If however, constantly condensation-capable sterilising agent vapour streams after, the ratio of the heat derived from the drops to the proportion of heat arising in the drops continues to fall and the drops become ever hotter. Finally, a sufficient number of drops can arise for a complete film to form. The drops or the condensate film can, in the extreme case, become so hot that no more vapour mass can precipitate. That is the case if the temperature of the condensate film is at a temperature which matches the vapour mass of the sterilising agent vapour flowing to the hollow body wall.

Condensate film temperatures of 120° C. have been observed. These high temperatures are however only reached for a short time as the wall material of the hollow body “HK” absorbs heat with a chronological delay to condensation and thus wall temperatures once again arise which allow vapour to condense. The duration of the condensation process is thus described by the mass flow dm/dt (mass per time) of the condensation-capable gas and the condensation conditions (the hollow body temperature, the concentration of the hydrogen peroxide vapour and the vapour density).

In practical operation of the vaporiser, temperatures of the condensation film of 120° C. with film thicknesses of 10 μm are observed. The typical duration of the condensation of vapour which leads to such high film temperatures and thicknesses is just 100 . . . 500 ms, whereby the condensation time to attain a film temperature is dependent on the density of the vapour passing over and, in this example, for 500 ms it typically lies at around 1 g/m̂3 and for 100 ms at around 3 g/m̂3. At lower temperatures of the condensate films (from around 50 to 100° C.) and lower condensate film thicknesses (around 1 to 5 μm), practicable condensation times for preforms and PET bottles stand at 300 to 5000 ms.

Advantageously, relatively large film thicknesses of the condensate film are selected, from 5 to 10 μm thick for hollow body disinfection, in which the hollow body “HK” is to be filled after disinfection or the preform is to be rapidly blow-moulded, for example if between the charging with disinfecting agent and the next work stage, for example the blowing out of disinfecting agent or the heating of the preform, there is a time of just 2 seconds. With condensate films this thick, temperatures are reached at which a sterilising level or the killing of Bacillus subtilis spores of 6 decades in 1 second is achieved. If a relatively a long time is available between the charging of the hollow body “HK” with sterilising agent and the next working stage, the condensate film does not have to be so thick. This is achieved by allowing a smaller mass of sterilising agent to flow in. With a condensate film of 1 pm, which is just still visible, the film temperatures are not so high and the sterilising agent or hydrogen peroxide needs a longer time to take effect. If left for 10 seconds to take effect, the killing of Bacillus subtilis spores of four decades is nonetheless achieved.

It has furthermore proved to be advantageous not to allow the gas or the sterilising agent vapour to cool isentropically, but for an almost exothermic expansion characteristic to be selected for the sterilising agent vapour when depressurising. Generally, the sterilising agent vapour would depressurised adiabatically or isentropically at the vaporiser outlet if it from this point carries out the depressurisation task at the cost of its internal energy and would consequently expand without heat exchange with the environment. The sterilising agent vapour would in contrast cool down isothermally if energy were constantly supplied to it corresponding to its temperature drop caused by the cooling. Both expansion characteristics are ideal characteristics and can only be approximately achieved. Certainly, a quasi-adiabatic cooling is achieved for rapidly depressurising vapours, which is evidenced by mist formation. Furthermore, for the vaporiser described below, by measuring the vapour temperature at the vapour outlet opening, it can be shown that a quasi-isothermal expansion can be achieved.

An expansion characteristic which proceeds close to the adiabatic has the drawback that the vapour cools down so much that it is then only slightly above the hollow body temperature or even has cooled so much before it reaches the hollow body wall, that condensation occurs before the hollow body wall which, in view of the factors stated above, is to be avoided (incomplete coating of condensate). In the case of the condensate formation occurring only at the hollow body wall, the low vapour temperature is not very serious as the condensation energy released in the condensate film is far greater than the vapour's energy associated with the heat capacity of the vapour.

In the case of condensate formation before the hollow body wall, however, condensate forms in the flowing vapour, due to which wet vapour or aerosol or mist forms. This vapour no longer condenses extensively as the drops have too much mass and this mass, driven by its impulses, hits the wall. Unsaturated vapour, i.e. vapour which does not contain any wet vapour, behaves differently during condensation than wet vapour. It behaves like a gas which flows everywhere by diffusion. If, then, on a surface the saturated vapour mass is exceeded and condensation is forced, in the first phase the first vapour molecules condense on the condensation cores present on the surface, i.e. the molecules which are the first to get there in the chronological course of the stream of molecules. At the places of the hollow body volume which are in the direct vicinity of these condensing gas molecules, i.e. the areas which are in front of the hollow body wall, a concentration gradient arises which drives more molecules to the locations of the already condensed molecules and lets condensation occur there. The release of condensation energy forming in the condensate is associated with the condensation. Small micro-droplets form, the temperature of which quickly rises compared to the vapour flowing past, so that the further condensation of molecules in the micro-drops now decreases or even stops, because in this micro-area, around the drops, the saturated vapour mass is high due to the temperature of the drops. Now the hollow body wall temperature which lies between two micro-drops, is lower than that of the micro-drops, and due to this a new condensation core arises which in turn allows a micro-drop to arise. This continues until the entire wall area is occupied by micro-drops. The size of the micro-drops and the duration from the start of condensation until complete condensate coating of the surface are dependent on the heat conduction property of the condensation area, the mass flow dm/dt of the vapour mass and the vapour density dm/dV.

Wet vapour precipitates on surfaces in a similar way to the behaviour of water jets from a shower head. Because of the large mass of drops (in comparison to the molecule masses), the drops simply follow their impulse. One must assume that, in comparison to unsaturated vapour, substantially more condensate mass is needed to cover a surface completely. Also, the complete coating of a surface with vapour takes far longer as condensate drops which formed before the hollow body wall in the vapour stream, have released part of their condensation energy to them due to impact with the air molecules of the hollow body and have therefore, already cooled, precipitated on the hollow body wall, and thus they, being on the hollow body wall, form condensation cores for the vapour phase (i.e. for the dry part) of the wet vapour and thus attract more vapour, and thus the formation of micro-droplets on still uncovered areas of the hollow body wall starts after a delay compared to the chronological course of the condensation of dry vapour.

An isothermal expansion characteristic can be achieved by supplying energy from outside to the vapour during the expansion. According to Kuchling (H. Kuchling, Taschenbuch der Physik, Carl-Hanser Verlag, 1999) for polytropic changes of state, the following functional connection between temperature and pressure is obtained:

$$\frac{{T}_{1}}{{T}_{2}} = {( \frac{{p}_{1}}{{p}_{2}} )}^{\frac{n - 1}{n}}$$

Here, T₁ is the temperature in state 1, for example before expansion, T₂ the temperature in state 2, for example after the expansion, both given in Kelvin degrees, p1 the pressure in state 1, in the invention described here a pressure in the range of 1.3 to 4 bar, for example 2.8 bar, p₂ the pressure in state 2, for example the depressurisation pressure of 1 bar and n designates the so-called polytropic exponent, which where n=1.32 describes the adiabatic depressurisation of hydrogen peroxide and water vapour and which where n=1 describes the isothermal depressurisation of both vapours. With isothermal depressurisation, the exponent of the formula becomes zero, whereby the temperature T₂, that is the temperature arising after the depressurisation, is the same as the temperature T₁.

During or after the depressurisation, the sterilising agent vapour is introduced into the hollow body “HK” to be disinfected, whereby it mixes with and further cools the gas in the hollow body “HK” to be disinfected. A mixture temperature arises which determines the saturated vapour density of the sterilising agent vapour before condensation. If energy is supplied to the sterilising agent vapour during the depressurisation, for example so that the depressurisation proceeds almost entirely isothermally, then hot sterilising agent vapour mixes with the atmosphere which is in the hollow body “HK” to be disinfected. If sterilising agent vapour at 120° C. is allowed to depressurise, the mixing of sterilising agent vapour with the air leads to a clear rise in the temperature of the gas atmosphere in the hollow body “HK” to be disinfected, whereby the saturated vapour mass of the sterilising agent vapour flowing into the hollow body “HK” is clearly increased, for example by a factor of 3 to 10, this being in comparison with the saturated vapour mass or density with adiabatic expansion of the sterilising agent vapour.

With adiabatic expansion, the temperature of the depressurising sterilising agent vapour would still lie only in the range of around 10 . . . 50° C., that of the isothermally depressurised sterilising agent vapour remains at the temperature before expansion, for example 110° c. to 140°. In the case of isothermal expansion, a mixture temperature of the sterilising agent with the air in the hollow body arises in the range of 40 . . . 60° C., whereby the mixture temperature is dependent on the hollow body volume and the streaming mass of sterilising agent. A temperature increase is thus achieved of around 20 to 40° C. In the case of adiabatic expansion, a mixture temperature arises which is only around 2° C.-5° C. higher than the gas temperature of the air before mixing.

For an isothermal or almost isothermal depressurisation, the vapour pipe 40 is designed as an isothermal pipe which provides just as much energy to the expanding sterilising agent vapour as it loses due to its expansion. This happens either by electrical heating (50) applied to the vapour pipe 40 (FIG. 2) or by heat conduction from the vaporiser 10 or by the sterilising agent vapour. In the event of heating by the sterilising agent vapour, the vapour pipe 40 has double walls with the formation of a heating duct through which the sterilising agent vapour is routed. In the event of heating by heat conduction, the vapour pipe 40 is made of a good heat conductor, e.g. of aluminium, and has a wall thickness of at least 2 mm, preferably a wall thickness of 3 mm or 4 mm. The internal diameter of the vapour pipe 40 lies for example in the range of 1 to 5 mm. The length of the vapour pipe 40 can be anything up to 200 mm. For vapour pipes 40, which are longer than 150 mm, a wall thickness of 4 mm is required for isothermal expansion.

One benefit of isothermal expansion is, inter alia, also that the depressurisation of the sterilising agent vapour can expediently be allowed to proceed with a not overly high starting pressure, whereby per unit of time only masses flow through the vapour pipe 40 which are of a size which can be well and completely heated in the vapour pipe 40 made in the form of an isothermal pipe during depressurisation.

Both expansion characteristics, isothermal and also adiabatic, are never completely achievable for most technical expansion processes, but many expansion processes can be very closely attributed to one or the other characteristic. If the isothermal expansion characteristic is to be achieved, sufficient energy must be supplied for as long as possible during expansion. If the adiabatic expansion characteristic is to be attained, the expansion must be as sudden as possible.

A further advantageous aspect of the invention arises where just enough sterilisation agent in vapour form is allowed to stream into the hollow body “HK” for the sterilisation agent not to condense during the streaming-in, whereby the hollow body “HK” is preheated by hollow body pre-heating, for example to 60□. Then, with isothermal expansion, a mixture temperature of over 80□ can be reached. In the hollow body “HK” and with a reaction time of 10 s, this vapour phase leads to a germ reduction of four decades for a Bacillus subtilis population. Advantageous in this method is that the quantity of hydrogen peroxide injected into the hollow body “HK” is small, whereby the quantity of sterilisation agent vapour generated in vaporiser 10 can be optimally used and a maximum number of hollow bodies “HK” can be treated with it. Furthermore, all parts of the system are charged with as little hydrogen peroxide as possible, which simplifies the handling of the hydrogen peroxide flowing out of the hollow bodies “HK” and reduces the load on the system parts.

The methods according to the invention of the above-described art can for example be carried out in a heating station for preforms upstream of the stretching and blowing machine for the blow-moulding of containers or bottles, whereby the aforesaid hollow body pre-heating for example is then upstream of the actual preform pre-heating. The methods according to the invention of the above-described art can be applied for example between the heating station for preforms and the stretching and blowing machine or after the stretching and blowing machine.

An advantageous embodiment of the invention exploits the use of two vaporisers in accordance with FIGS. 3-5, whereby an additional vaporiser 10.1 functions as a pre-vaporiser in which the sterilising agent is introduced in a measured manner via the metering unit 20 and/or the metering pump 20.1 and in which a large quantity of sterilising agent vapour is produced which is then passed on cyclically during the period t2−t1 to at least one vaporiser 10, whereby the vaporiser 10 in turn serves as a injection vaporiser for the sterilising agent vapour which is introduced by the vaporiser 10 via the vapour pipe 40 into the hollow body “HK” to be disinfected. To pass on the sterilising agent vapour from the vaporiser 10.1 into the vaporiser 10, a valve 60, which is in the overflow pipe 70, is opened for a period t2−t1, which is illustrated in FIG. 5 by the diagram “Valve 60”. By opening the valve 60, the pressure p₂ builds in vaporiser 10. At the same time, the pressure in the vaporiser 10.1 falls from p1 to a lower value (FIG. 5, diagrams “Vaporiser 10” and “Vaporiser 10.1”).

The sterilising agent vapour which is stored in the vaporiser 10 can be used one or more times, to be let via the valve 80 (discharge valve) and the vapour pipe 40 after this valve in the direction of the stream of vapour, into the hollow body “HK” to be disinfected. To do this, the valve 80 simply needs to be opened for a certain time as shown in FIG. 5 by the switching status 1 of the diagram “Valve 80”. The sterilising agent vapour streams either very slowly through the discharge valve 80 into the hollow body “HK”, whereby at the outlet of valve 80 the flow of the sterilising agent vapour is restricted by a diaphragm, or alternatively the sterilising agent vapour streams through the vapour pipe 40 formed as an isothermal pipe into the hollow body “HK”. The vapour pipe 40 formed as an isothermal pipe supplies the expanding sterilising agent vapour with precisely the same amount of energy as it is losing by its expansion. This happens in the previously described manner either by the electrical heating 50 (not shown in FIGS. 3 and 4) applied to the vapour pipe 40 or by heat conduction from the vaporiser 10 or heating with the sterilising agent vapour. In the event of heating by the sterilising agent vapour, the vapour pipe 40 again has double walls, and the sterilising agent vapour is routed through a duct between the two walls. In the case of heat conduction, the vapour pipe 40 is made for example of aluminium, and has a wall thickness of at least 2 mm, preferably 3 mm-4 mm. The internal diameter of the vapour pipe 40 can be around 1 to 5 mm, for a length of up to 200 mm. For vapour pipes 40, which are longer than 150 mm, a wall thickness of 4 mm is required for isothermal expansion.

If the vaporiser 10 is filled to p₂, the vapour can be used for a number of hollow bodies “HK” before the vaporiser 10 is re-filled. Likewise, a vapour cushion produced in 10.1 can be used a number of times before disinfecting agent is injected once again into the vaporiser 10.1 to increase the pressure to p1 again.

As shown in FIG. 4, where the vaporiser 10.1 (pre-vaporiser) is used, the vaporiser 10 can be made with a reduced volume, in particular in such a way that the volume of the vaporiser 10 is smaller than the volume of the vaporiser 10.1. Inter alia, in systems with a number of treatment positions, to each of which a vaporiser 10 is allocated, and with at least one pre-vaporiser 10.1 common to a number of treatment positions, this has the advantage that the vaporiser 10 can be made with reduced dimensions for a compact design.

In a further advantageous embodiment of the invention, in accordance with FIG. 6, a number of vaporisers 10 are run on one vaporiser 10.1 (pre-vaporiser). A number of arrangements according to FIG. 6 can be disposed next to each other, for example on a rotor (carousel). In this way, a circular process is possible in which the hollow bodies “HK” to be disinfected are passed on to the rotor and the valves 60 and 80 are opened and closed under process control in a process management adapted to the circular process. Naturally, in doing so, the metering unit 20 and a metering valve allocated to this metering unit 20 and/or the metering pump 20.1 are controlled in the necessary way. Here, it is also possible to provide two or more, as two metering units 20 and/or metering pumps 20.1, to one vaporiser 10.1, as shown in FIG. 7.

In a further advantageous embodiment of the invention, at least two vaporisers 10.1 (pre-vaporisers) are run on one vaporiser 10 each. Then the vaporisers 10.1 can be used alternately to feed the associated vaporiser 10, which facilitates the production of dry sterilising agent vapour, and in particular because there are cycles in which at least one of the vaporisers 10.1 is used only to form the vapour phase and this vapour phase can be precisely controlled because in this cycle no sterilising agent vapour flows out of this vaporiser 10.1 to the vaporisers 10.

In a further advantageous embodiment of the invention, the ratio of the volume of vaporiser 10.1 to the volume of vaporiser 10 is at least two and even more advantageously at least five and even more advantageously at least ten.

In a further advantageous embodiment of the invention, the vaporisers 10.1 and 10 are operated at different temperatures. Advantageously the temperature of the vaporiser 10.1 is at least 5° C. more than the temperature of the vaporiser 10.

In a further advantageous embodiment of the invention, the vaporisers 10 and/or 10.1 are evacuated cyclically by a vacuum device. This has proved to be particularly advantageous in operating breaks or during conditioning procedures.

In a further advantageous embodiment of the invention, the injection interval t4−t3, in which the valve 80 is opened, to feed the sterilising agent vapour into a hollow body “HK” to be disinfected, is split into at least two injections, whereby the valve 80 is closed for a defined time between the two injections. For the isothermal expansion in particular, this has benefits in the case where the vapour pipe 40 formed as an isothermal pipe is heated by heat conduction from the vaporiser 10.

In a further advantageous embodiment of the invention, the mass flow dm/dt of the sterilising agent vapour and the speed of the removal of the isothermal pipe from the hollow body “HK” to be disinfected are adapted in such a way that, when the isothermal pipe is pulled out, a uniform thin condensate layer is produced.

Particularly advantageous is an embodiment of the invention in which a particular quantity of hydrogen peroxide is vaporised in the particular vaporiser 10.1 (pre-vaporiser), for example a quantity which corresponds to the saturated vapour mass at 140° C. Then, with 20% H2O2, a pressure of 3.1 bar would arise, whereby after complete vaporisation this sterilising agent vapour streams in part into the second vaporiser. For this, an adiabatic depressurisation characteristic can be selected because the vaporiser 10 is operated at a temperature which is only slightly lower than the temperature of the first vaporiser, for example 0 to 30° C. lower. The depressurisation at the outlet of the second vaporiser (vaporiser 10) must however (ideally) occur isothermally (polytropic coefficient of 1). A polytropic coefficient of 1.1. is however still not sufficient.

On the basis of the equation for polytropic changes of state

$$\frac{{T}_{1}}{{T}_{2}} = {( \frac{{V}_{2}}{{V}_{1}} )}^{n - 1}$$

for the adiabatic change of state (polytropic coefficient n is the same as the adiabatic exponent k and for water vapour and hydrogen peroxide vapour approx. 1.32) when the sterilising agent vapour flows from one vaporiser 10.1 into the other vaporiser 10 with a volume increase of 50% (total of the volumes of vaporisers 10 and 10.1 is 3, total of the volume of the vaporiser 10.1 is 2) in state 2 after said flow, a temperature T2 arises, which is around 13% lower than the temperature T1 in state 1 before said flow (for example T₁=413 K and T₂=363 K). If the volume increase is even lower, for example if the vaporiser 10 has only one quarter of the volume of the first vaporiser, then the temperature T₂ in state 2 even stands at 93% of the starting value T₁.

Because of the relatively low temperature loss when a certain quantity of sterilising agent vapour flows from the vaporiser 10.1 into the vaporiser 10, said temperature loss occurring with the sterilising agent vapour in the vaporiser 10, if its volume is only a fraction of the volume of 10.1, the gas which has flowed into the vaporiser 10 can quickly be brought back to the temperature of the vaporiser 10, for example in around 2 to 5 seconds.

Common to all embodiments is that sterilising agent vapour is provided as dry sterilising agent vapour for introduction into the relevant hollow body “HK” in vaporiser 10 and the introduction without an additional carrier gas and/or occurs such that the sterilising agent vapour flows in a dry or at most in a saturated state into the particular hollow body “HK” so that the condensate formation occurs only or at least in the main only on the hollow body wall.

If there are a number of treatment stations for the treatment of the hollow bodies “HK”, for example on a rotor around a vertical machine axis, then at least one vaporiser 10 is allocated to each treatment station or group of treatment stations. Finally, it should be noted that the method can also be used in a similar way for the external vapour deposition on hollow body surfaces to achieve an external disinfection or sterilisation. In an application and method of this kind, one or more hollow bodies are put in a receiving body or a sterilising chamber and the sterilising vapour is then fed into the space between the hollow body and receiving body. The aforesaid process step and parameters are carried out or performed in an analogous manner.

### REFERENCE DRAWING LIST

10, 10.1 Vaporiser
20 Metering unit
20.1 Metering pump
30 Discharge opening
40 Vapour pipe
50 Heating
60 Valve
70 Overflow pipe
80 Valve (discharge valve)

1-19. (canceled)

20. A method for sterilizing a hollow body by using a sterilizing agent, said sterilizing agent being introduced from a vaporizer, in vaporized form, into said hollow body, said hollow body being at ambient pressure, said method comprising supplying said sterilizing agent in said vaporizer as a dry sterilizing agent vapor, said dry sterilizing agent vapor comprising vapor with a sterilizing agent quantity or vapor density that is at most equal to saturated vapor density such that upon introduction of said sterilizing agent vapor into said hollow body and upon subsequent expansion of said sterilizing agent vapor from a pressure in said vaporizer to ambient pressure, condensate formation occurs substantially only on a surface of said hollow body.

21. The method of claim 20, further comprising introducing sterilizing agent vapor into said hollow body without a carrier gas.

22. The method of claim 20, further comprising isothermally depressurizing said sterilizing agent vapor introduced into said hollow body.

23. The method of claim 20, further comprising selecting said sterilizing agent to be an aqueous hydrogen peroxide solution, wherein pressure of said sterilizing agent vapor in said vaporizer depends on temperature in said vaporizer and on concentration of hydrogen peroxide in said sterilizing agent in accordance with the following table:

| T = 120° C. | | T = 130° C. | |
|---|---|---|---|
| KN [%] | P [bar] | KN [%] | P [bar] |
| 10 | 1.78 | 10 | 2.51 |
| 20 | 1.65 | 20 | 2.32 |
| 30 | 1.48 | 30 | 2.09 |
| T = 140° C. | | T = 150° C. | |
| KN [%] | P [bar] | KN [%] | P [bar] |
| 10 | 3.36 | 10 | 4.48 |
| 20 | 3.1 | 20 | 4.14 |
| 30 | 2.79 | 30 | 3.73 |

where T is the temperature of one of the vaporizer and the wall temperature of the vaporizer, KN is hydrogen peroxide concentration in the sterilizing agent as a percentage by weight and P is pressure in the vaporizer.

24. The method of claim 20, wherein said vapor density of said sterilizing agent vapor in said vaporizer, with a temperature inside said vaporizer of around 120° C., is no more than 1.7 kg/m³, and/or wherein said sterilizing agent vapor, after introduction thereof in said hollow body at an ambient temperature of 20° C., has a vapor density of up to 17 g/m³.

25. The method of claim 20, wherein a thickness of condensate precipitated on said hollow body is approximately 10 μm and/or duration of treatment of said hollow body is no more than two seconds, and wherein a reaction time of said condensate from said sterilizing agent is around 10 seconds.

26. The method of claim 20, further comprising pre-heating said hollow body to a temperature between 55 C and 65 C.

27. The method of claim 20, further comprising heating said hollow body, due to said condensate formation, to a temperature of up to 120° C. for a time sufficient to automatically activate said sterilizing agent.

28. The method of claim 20, further comprising measuring said sterilizing agent, introducing said sterilizing agent into a pre-vaporizer, vaporizing said sterilizing agent in said pre-vaporizer to form said sterilizing agent vapor, adiabatically increasing pressure of said sterilizing agent vapor, and introducing said sterilizing agent vapor into said vaporizer, whereby a quantity of sterilizing agent introduced is selected such that said quantity is no more than saturated vapor mass at a temperature of one of said pre-vaporizer and said vaporizer.

29. The method of claim 28, further comprising monitoring decomposition rate of said sterilizing agent in said sterilizing agent vapor by measuring vapor pressure of at least one of said pre-vaporizer and vaporizer.

30. The method of claim 20, wherein introduction of said sterilizing agent vapor into said particular hollow body occurs in at least two consecutive and chronologically separate treatment steps.

31. The method of claim 20, said method further comprising placing said hollow body in a sterilizing chamber, and introducing said sterilizing agent into a space between an external wall area of said hollow body and an internal wall area of said sterilizing chamber, thereby sterilizing said external wall area of said hollow body.

32. The method of claim 20, further comprising selecting said sterilizing agent to be hydrogen peroxide.

33. The method of claim 20, further comprising selecting said sterilizing agent to be peracetic acid.

34. A method for sterilizing a hollow body using a sterilizing agent, said sterilizing agent being introduced from a vaporizer in vaporized form into said hollow body, said hollow body being at ambient pressure, said method comprising supplying said sterilizing agent in said vaporizer as a dry sterilizing agent vapor, said dry sterilizing agent vapor comprising one of a sterilizing agent quantity and vapor density that is at most equal to a saturated vapor quantity or density, and introducing said sterilizing agent vapor into said hollow body without a carrier gas.

35. The method of claim 34, wherein upon introducing said sterilizing agent vapor into said hollow body, and upon subsequent expansion of said sterilizing agent vapor from a pressure in said vaporizer to ambient pressure, condensate formation in vapor flow takes place primarily only on a surface of said hollow body.

36. An apparatus for sterilizing a hollow body using a sterilizing agent, said apparatus comprising a treatment station comprising a vaporizer for introducing said sterilizing agent, in vaporized form and with a vapor pressure greater than ambient pressure, into said hollow body, which is at ambient pressure, wherein said vaporizer is configured to supply said sterilizing agent as a dry sterilizing agent vapor in which a sterilizing agent quantity or vapor density is at most equal to saturated vapor quantity or density, and to introduce said sterilizing agent vapor into said hollow body without a carrier gas.

37. The apparatus of claim 36, further comprising at least two pre-vaporizers allocated to said vaporizer, said two pre-vaporizes operating alternately.

38. The apparatus of claim 36, wherein said vaporizer comprises an isothermal vapor pipe for said introducing said sterilizing agent vapor into said hollow body with an isothermal depressurization characteristic.

39. The apparatus of claim 36, wherein areas coming into contact with said sterilizing agent or sterilizing agent vapor comprise a coating that reduces decomposition of said sterilizing agent.

40. The apparatus of claim 36, further comprising a plurality of treatment stations for sterilizing of hollow bodies, and a vaporizer allocated to each set of one or more treatment stations.
